(19) United States
(12) Patent Application Publication
(10) Pub. No.: US 2009/0305983 A1
(43) Pub. Date: Dec. 10, 2009

(54) Curable Bone Cement

(75) Inventors: Jackie Y. Ying, Nanos (SG); Shona Pek, Nanos (SG); Motoichi Kurisawa, Nanos (SG); Joo Eun Chung, Nanos (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Agency for Science, Technology and Research, Centros (SG)

(21) Appl. No.: 12/330,436

(22) Filed: Dec. 8, 2008

Related U.S. Application Data
(63) Continuation-in-part of application No. 12/280,777, filed on Jan. 14, 2009.

(51) Int. Cl. A61K 38/18; A61P 19/00

(52) U.S. Cl. 514/12

(57) ABSTRACT

The present invention describes a curable bone cement. The cement comprises a curable polymeric binder and a filler, and is capable of curing without substantial evolution of heat on exposure to a curing agent. The binder comprises phenol groups which are capable of reacting in order to cure the cement.

### RELATED APPLICATION INFORMATION

This application is a continuation in part of U.S. patent application Ser. No. 12/280,777, filed on Aug. 26, 2008, which is the U.S. National Phase entry under 35 U.S.C. § 371 and claims the benefit of International Application No. PCT/SG2006/000039, filed Feb. 27, 2006. Each of these applications are hereby incorporated by reference in their entirety.

### TECHNICAL FIELD

The present invention relates to a curable composition for use in bone cement applications.

### BACKGROUND OF THE INVENTION

Many clinical procedures such as maxillofacial surgery and osteochondral surgery require the use of bone cements to fill bone defects and deficiencies. Otherwise, the bone defects and deficiencies would not heal properly, preventing the return of normal function. Various synthetic bone substitutes have been developed for this purpose, some of which have been produced in an injectable form, so as to enable minimally invasive surgery. The main use of injectable bone substitutes include spinal fusion, bone and joint defects, osteoporotic fractures, revision surgery and vertebroplasty. A common disadvantage of injectable bone substitutes is that they generate heat during the process of curing. This heat has the potential to damage surrounding tissue.

There is therefore a need for a curable bone substitute that does not generate heat when curing.

### OBJECT OF THE INVENTION

It is the object of the present invention to overcome or substantially ameliorate at least one of the above disadvantages.

### SUMMARY OF THE INVENTION

Disclosed herein is a curable bone cement comprising a curable binder and a filler, wherein the cement (and/or the binder) is capable of curing without substantial evolution of heat. The cement may be capable of curing on exposure to (e.g. combination with, mixing with or addition of) a curing agent. The curing agent may be a reagent or may be a catalyst. The binder and the filler may be biocompatible. The curing agent may be biocompatible.

The binder may be crosslinkable without substantial evolution of heat. It may be a polymeric or oligomeric binder. It may be crosslinkable by means of an oxidant, e.g. a mild oxidant. It may comprise —C₆R′₄OR groups (i.e. phenol groups), wherein R and each R′ may independently be hydrogen, an alkyl group, an aryl group or an acyl group, and R′ may also be OH. Each R′ may be the same as or different to each other R′, provided that at least one R′, for example an R′ ortho to the OR group, is hydrogen. R and R′ may be such that one —C₆R′₄OR group is capable of oxidatively coupling with another —C₆R′₄OR group. The —C₆R′₄OR groups may be for example —C₆H₄OH groups. The binder may comprise a combination, a complex, a reaction product or a conjugate, of a polymeric species and a phenolic species. The phenolic species may be a polyphenol. Suitable phenolic species include tyramine, catechin, epicatechin, gallic acid and epigallocatechin gallate (EGCG), as well as mixtures of any two or more thereof. The polymeric species may be a biopolymer or a derivative thereof. It may be for example hyaluronic acid, a polyamine or a polypeptide, such as gelatin and/or collagen. The filler may be an apatite filler, for example hydroxyapatite, carbonated apatite, fluoroapatite, or any form of modified apatite or a combination of several types of apatite in any proportion, or may be some other mineral filler for example silica, alumina, zirconia, calcium phosphate, talc, calcium carbonate, mica.

In a first aspect of the invention there is provided a curable bone cement comprising a curable polymeric binder and a filler, wherein the cement is capable of curing without substantial evolution of heat on exposure to a curing agent, said binder comprising phenol groups which are capable of reacting in order to cure the cement. The phenol groups may be capable of oxidatively coupling in order to cure the polymeric binder. The phenol groups may be —C₆R′₄OR groups, wherein R and each R′ are independently hydrogen, an alkyl group, an aryl group or an acyl group, and R′ may also be OH, and each R′ is the same as or different to each other R′, provided that at least one R′, for example an R′ ortho to the OR group, is hydrogen, and wherein R and R′ are such that one —C₆R′₄OR group is capable of oxidatively coupling with another —C₆R′₄OR group.

At least some of the —C₆R′₄OR groups may be —C₆H₄OH groups. The binder may comprise for example a hyaluronic acid-tyramine (HA-Tyr) conjugate, a gelatin-Tyr conjugate or a hyaluronic acid-epigallocatechin gallate (HA-EGCG) conjugate. The filler may comprise a mineral filler, for example silica, alumina, zirconia, talc, an apatite or a mixture of any two or more of these. The filler may additionally or alternatively comprise particles capable of releasing a drug, a protein and/or a growth factor. The particles may be controlled release particles. Such particles may be useful for enhancing healing of the bone or of tissue surrounding the bone. Examples of suitable apatite fillers include hydroxyapatite, carbonated apatite, fluoroapatite, or any form of modified apatite or a combination of two or more types of apatite in any proportion. An example of a suitable apatite filler is a mixture of hydroxyapatite (HAP) and carbonated apatite (CAP). The curing agent may be selected so that the bone cement is capable of curing in acceptable time at the temperature of use (e.g. at the body temperature into which the bone cement is injected). The bone cement may be capable of curing in between about 10 seconds and about 30 minutes, or between about 20 seconds and 1 minute on exposure to the curing agent at the body temperature of a patient in which the cement is cured. The curing agent may comprise an oxidant. The curing agent may be an agent for oxidative coupling of phenolic groups. The curing agent may be a mild oxidant so that curing of the cement may be accomplished without substantial evolution of heat. The curing agent may comprise an enzyme, e.g. a peroxidase. It may comprise a peroxide. It may comprise a combination of a peroxide and an enzyme e.g. a peroxidase such as horse radish peroxidase (HRP). For example, the curing agent may comprise hydrogen peroxide and horse radish peroxidase. Other suitable curing agents comprise glutathione peroxidase, myeloperoxidase, tyrosinase or laccase in combination with or without a peroxide.

The bone cement may additionally comprise one or more further component such as collagen, a silicate, a protein (e.g. growth factor) and platelets.

The bone cement may be injectable. It may be in the form of a paste, or a slurry or some other viscous preparation. It may show shear thinning (pseudoplastic) rheology. It may show plastic rheology i.e. it may exhibit a finite yield stress. Once mixed with the curing agent, the bone cement may be injectable. It may be in the form of a paste, or a slurry or some other viscous preparation.

In an embodiment the curable bone cement comprises:
a conjugate of hyaluronic acid with a compound selected from the group consisting of tyramine, catechin, epicatechin, gallic acid and epigallocatechin gallate, and mixtures of any two or more thereof, and
an apatite filler,
   whereby the cement is curable on exposure to a peroxide and a peroxidase enzyme without substantial evolution of heat.

In another embodiment, the curable bone cement comprises a hyaluronic acid-tyramine (HA-Tyr) conjugate and an apatite filler, whereby the cement is curable on exposure to hydrogen peroxide and horse radish peroxidase without substantial evolution of heat.

In another embodiment the curable bone cement comprises:
a conjugate of a polyamine or a polypeptide such as gelatin and/or collagen with a compound selected from the group consisting of tyramine, catechin, epicatechin, gallic acid and epigallocatechin gallate, and mixtures of any two or more thereof, and
an apatite filler,
   whereby the cement is curable on exposure to a peroxide and a peroxidase enzyme without substantial evolution of heat.

The curable bone cement may contain a mixture of gelatin-Tyr, HA-Tyr and/or an apatite filler.

There is also provided the use of a curable binder and a filler for the manufacture of a bone cement for use in repairing bones, said binder comprising phenol groups with at least one hydrogen atom attached to the aromatic ring thereof.

There is also provided a kit comprising a curable bone cement according to the first aspect and a curing agent, whereby said curing agent is capable of causing the curable bone cement to cure without substantial evolution of heat. The curing agent may comprise more than one component, optionally two components. The components may be added separately to the curable bone cement in order to induce curing. Alternatively they may be added together to the curable bone cement in order to induce curing. The ratio of the bone cement to the curing agent in the kit may be such that, when the bone cement and the curing agent of the kit are combined in said ratio, the bone cement is capable of curing in between about 10 seconds and about 30 minutes at the body temperature of a patient. There is further provided a catalysed bone cement comprising the curable bone cement combined with the curing agent.

In a second aspect of the invention there is provided a process for making a curable bone cement comprising combining a solution of a curable binder with a filler, and optionally with one or more further component such as collagen, a silicate, a protein (e.g. growth factor) and platelets, said binder comprising phenol groups which are capable of reacting in order to cure the cement. The curable binder and the filler may be as described above. Thus for example the filler may comprise an apatite or a mixture of two or more apatites. The step of combining may comprise preparing a solution of the curable binder. It may comprise combining the solution of the curable binder with the filler.

The process may also comprise the step of making the curable binder. This may comprise coupling a phenolic species with a polymeric species. The polymeric species may be a biopolymer, e.g. hyaluronic acid, or a derivative thereof. It may be a polyamine or a polypeptide, e.g. gelatin or collagen. The phenolic species may comprise one or more —C₆R′₄OR groups. It may or may not comprise an amine functional group.

In an embodiment, the process comprises combining a solution of a curable binder, such as a hyaluronic acid-tyramine (HA-Tyr) conjugate, with an apatite filler, and optionally with one or more further component such as collagen, a silicate, a protein (e.g. growth factor) and platelets.

In another embodiment the process comprises:
coupling a phenolic species with a polymeric species to form a curable binder; and
combining a solution of the curable binder with a filler, and optionally with one or more further components such as collagen, a silicate, a protein (e.g. growth factor) and platelets, said binder comprising phenol groups which are capable of reacting in order to cure the cement.

The invention also provides a curable bone cement when made by the process of the second aspect.

There is also provided a process for making a catalysed bone cement comprising providing a curable bone cement according to the first aspect and exposing (e.g. combining, mixing or adding) said curable bone cement to a curing agent, whereby said curing agent is capable of causing the curable bone cement to cure without substantial evolution of heat. The step of providing the curable bone cement may comprise preparing said curable bone cement, for example by the process of the second aspect of the invention.

In a third aspect of the invention there is provided a method for curing a bone cement, said bone cement comprising a curable binder and a filler, said binder comprising phenol groups which are capable of reacting in order to cure the cement, said method comprising:
exposing the curable bone cement to a curing agent to form a catalysed bone cement; and
curing the catalysed bone cement without substantial evolution of heat.

The curable binder, the filler and the curing agent may be as described above. Thus for example the filler may comprise an apatite or a mixture of two or more apatites and the curing agent may comprise a peroxide and a peroxidase enzyme. The process may comprise the step of injecting the bone cement into a patient, or otherwise locating the bone cement in and/or on the bone of a patient. This step may be conducted before the step of curing the catalysed bone cement. The curable bone cement and the curing agent may be used in non-toxic amounts in the patient. The curing agent may comprise more than one component, optionally two components. The components may be added separately to the curable bone cement in order to induce curing. Alternatively they may be added together to the curable bone cement in order to induce curing.

The invention also provides a cured bone cement when made by the process of the third aspect of the invention.

In a fourth aspect of the invention there is provided a method for repairing a bone in a patient comprising:
combining a curable bone cement comprising a curable binder and a filler with a curing agent to form a catalysed bone cement, said binder comprising phenol groups which are capable of reacting in order to cure the cement,
injecting said catalysed bone cement onto and/or into said bone; and
curing the catalysed bone cement on and/or in the bone without substantial evolution of heat.

The curable binder, the filler and the curing agent may be as described above. Thus for example the filler may comprise an apatite or a mixture of two or more apatites and the curing agent may comprise a peroxide and a peroxidase enzyme.

In a fifth aspect of the invention there is provided a curable bone cement comprising a curable polymeric binder and a filler, wherein the cement is capable of curing without substantial evolution of heat on exposure to a curing agent, said binder comprising a reaction product of:

an aminofunctional polymer, and

an alkanoic acid bearing a phenolic group, said phenolic group in the binder being capable of reacting in order to cure the cement.

The following options may be used in conjunction with the fifth aspect, either individually or in any suitable combination.

The phenolic group may be —C₆H₄OH. The alkanoic acid may be terminally substituted with a 4-hydroxyphenyl group.

The alkanoic acid may be a C2 to C6 straight chain alkanoic acid.

The alkanoic acid bearing a phenolic group may be 3-(4-hydroxyphenyl)propionic acid.

The aminofunctional polymer may be selected an aminofunctional polysaccharide, a polyamine or a polypeptide, or may be a mixture of any two or all of these. The aminofunctional polymer may be a protein. The protein may be gelatine.

The curing agent may comprise an oxidant. It may comprise a peroxidase enzyme and a peroxide. It may comprise hydrogen peroxide and horse radish peroxidase.

The curable bone cement may be capable of curing to a solid in between about 10 seconds and about 30 minutes without substantial evolution of heat on exposure to the curing agent at the body temperature of a patient in which the cement is cured.

The filler may comprise a mineral filler. It may comprise an apatite or a mixture of two or more apatites.

The curable bone cement of claim 1 additionally comprising a second binder, said second binder being a reaction product of:

a carboxylic acid functional polymer, and

an alkylamine bearing a second phenolic group said second phenolic group in the second binder being capable of reacting in order to assist in the curing of the cement.

The carboxylic acid functional polymer of the second binder may be a polysaccharide. The polysaccharide may be hyaluronic acid. The alkylamine of the second binder may be a primary amine terminally substituted with a 4-hydroxyphenyl group. It may be a C1 to C6 primary amine terminally substituted with -hydroxyphenyl group. It may be tyramine

The second binder may be capable of reacting in order to assist in the curing of the cement under the same conditions as are required for curing of the binder which comprises a reaction product of an aminofunctional polymer and an alkanoic acid bearing a phenolic group.

The curable bone of the fifth aspect may additionally comprise at least one further component selected from the group consisting of collagen, a silicate, a protein and platelets. The protein may be a growth factor.

In an embodiment there is provided a curable bone cement comprising a curable polymeric binder and a filler, wherein the cement is capable of curing without substantial evolution of heat on exposure to a curing agent, said binder comprising a reaction product of gelatine and 3-(4-hydroxyphenyl)propionic acid.

In another embodiment there is provided a curable bone cement comprising a curable polymeric binder and an apatite filler, wherein the cement is capable of curing without substantial evolution of heat on exposure to a curing agent, said binder comprising a reaction product of gelatine and 3-(4-hydroxyphenyl)propionic acid.

In another embodiment there is provided a curable bone cement comprising a curable polymeric binder, a second binder and an apatite filler, wherein the cement is capable of curing without substantial evolution of heat on exposure to a curing agent, said binder comprising a reaction product of gelatine and 3-(4-hydroxyphenyl)propionic acid and said second binder comprising a reaction product of hyaluronic acid an tyramine.

In another embodiment there is provided a curable bone cement comprising a curable polymeric binder, an apatite filler and a growth factor, wherein the cement is capable of curing without substantial evolution of heat on exposure to a curing agent, said binder comprising a reaction product of gelatine and 3-(4-hydroxyphenyl)propionic acid

In a sixth aspect of the invention there is provided a catalysed bone cement comprising the curable bone cement of the fifth aspect combined with the curing agent.

The bone cement may be injectable. It may be in the form of a paste.

In an embodiment there is provided an injectable catalysed bone cement comprising:
a curable bone cement comprising a curable polymeric binder and an apatite filler, wherein the cement is capable of curing without substantial evolution of heat on exposure to a curing agent, said binder comprising a reaction product of gelatine and 3-(4-hydroxyphenyl)propionic acid; and
a curing agent comprising horse radish peroxidase and hydrogen peroxide.

In another embodiment there is provided an injectable catalysed bone cement comprising:
a curable bone cement comprising a curable polymeric binder, a second binder and an apatite filler, wherein the cement is capable of curing without substantial evolution of heat on exposure to a curing agent, said binder comprising a reaction product of gelatine and 3-(4-hydroxyphenyl)propionic acid and said second binder comprising a reaction product of hyaluronic acid an tyramine; and
a curing agent comprising horse radish peroxidase and hydrogen peroxide.

In a seventh aspect of the invention there is provided a process for making a curable bone cement comprising combining a curable polymeric binder and a filler, said binder comprising a reaction product of an aminofunctional polymer and an alkanoic acid bearing a phenolic group, said phenolic group in the binder being capable of reacting in order to cure the cement without substantial evolution of heat on exposure to a curing agent at the body temperature of a patient in which the cement is cured.

The following options may be used in conjunction with the seventh aspect, either individually or in any suitable combination.

The curable polymeric binder may be dissolved in a solvent prior to said combining. The solvent may be an aqueous solvent.

A second binder may be combined with the binder comprising a reaction product of an aminofunctional polymer and an alkanoic acid bearing a phenolic group, said second binder being a reaction product of:

a carboxylic acid functional polymer, and

an alkylamine bearing a second phenolic group,

said second phenolic group in the second binder being capable of reacting in order assist in the curing of the cement.

In an embodiment there is provided a process for making a curable bone cement comprising combining a curable polymeric binder, a second binder and a filler, said binder comprising a reaction product of an aminofunctional polymer and an alkanoic acid bearing a phenolic group and said second binder being a reaction product of a carboxylic acid functional polymer and an alkylamine bearing a second phenolic group, said phenolic groups in the binder and in the second binder being capable of reacting in order to cure the cement without substantial evolution of heat on exposure to a curing agent at the body temperature of a patient in which the cement is cured

In an eighth aspect of the invention there is provided a method for curing a curable bone cement, said method comprising:
exposing the curable bone cement to a curing agent to form a catalysed bone cement; and
curing the catalysed bone cement without substantial evolution of heat;
   wherein the bone cement comprises a curable polymeric binder and a filler, said binder comprising a reaction product of an aminofunctional polymer and an alkanoic acid bearing a phenolic group, said phenolic group in the binder being capable of reacting in order to cure the cement.

The curing agent may comprise an oxidant. The curing agent may comprise more than one component, optionally two components. The components may be added separately to the curable bone cement in order to induce curing. Alternatively they may be added together to the curable bone cement in order to induce curing. It may comprise a peroxidase enzyme and a peroxide.

The method may additionally comprise the step of injecting the bone cement into a patient before the step of curing the catalysed bone cement.

In an embodiment there is provided a method for curing a curable bone cement, said method comprising:
exposing the curable bone cement to a curing agent to form a catalysed bone cement; and
curing the catalysed bone cement without substantial evolution of heat;
   wherein the bone cement comprises a curable polymeric binder and an apatite filler, said binder comprising a reaction product of gelatine and 3-(4-hydroxyphenyl)propionic acid, and said curing agent comprising horseradish peroxidase and hydrogen peroxide.

In a ninth aspect of the invention there is provided a method for at least partially repairing a bone in a patient comprising:
combining a curable bone cement with a curing agent to form a catalysed bone cement,
injecting said catalysed bone cement onto and/or into said bone; and
curing the catalysed bone cement on and/or in the bone without substantial evolution of heat;
   wherein the bone cement comprises a curable polymeric binder and a filler, said binder comprising a reaction product of an aminofunctional polymer and an alkanoic acid bearing a phenolic group, said phenolic group in the binder being capable of reacting in order to cure the cement.

### BRIEF DESCRIPTION OF THE DRAWINGS

A preferred form of the present invention will now be described by way of example with reference to the accompanying drawings wherein:

FIG. 1 shows micrographs of bone injected with a bone cement according to the present invention, with staining with (a) H and E, (b) ALP and NFR, and (c) VK and NFR for cement 1 of the example (HA solution plus curing agent) (control) 5 weeks after injection;

FIG. 2 shows micrographs of bone injected with a bone cement according to the present invention, with staining with (a) H and E, (b) ALP and NFR, and (c) VK and NFR for cement 2 of the example (HA solution and apatite powders, plus curing agent) 5 weeks after injection;

FIG. 3 shows micrographs of bone injected with a bone cement according to the present invention, with staining with (a) H and E, (b) ALP and NFR, and (c) VK and NFR for cement 3 of the example (HA solution and apatite powders, and collagen solution, plus curing agent) 5 weeks after injection;

FIG. 4 shows micrographs of bone injected with a bone cement according to the present invention, with staining with (a) H and E, (b) ALP and NFR, and (c) VK and NFR for cement 4 of the example (HA solution, and pre-mixed collagen-apatite solution, plus curing agent) 5 weeks after injection;

FIG. 5 shows a representative crosslinked structure according to the present invention;

FIG. 6 shows a scheme for making a HA-dialkyl acetal conjugate;

FIG. 7 shows a scheme for making a HA-EGCG conjugate;

FIG. 8 shows a scheme showing synthesis of apatite/Gtn-HPA cement;

FIG. 9 shows a DSC curve associated with the setting of apatite/Gtn-HPA cement;

FIG. 10 a graph illustrating the effect of H₂O₂ concentration on the (▴) G′, (♦) σ_{y}, (▪) E_{c} and (*) swelling ratio of apatite/Gtn-HPA cements with 0.4 g/ml of apatite and 0.15 units/ml of HRP;

FIG. 11 shows a graph illustrating the effect of apatite concentration on the (▴) G′, (♦) σ_{y}, and (▪) E_{c} of apatite/Gtn-HPA cements with 16.5 mM of H₂O₂ and 0.15 units/ml of HRP;

FIG. 12 shows a comparison of various apatites, trabecular bone and the bone cement of the present invention;

FIG. 13 shows IR spectra of apatites, the bone cement of the invention and trabecular bone;

FIG. 14 shows the change in gelation time with HRP concentration;

FIG. 15 is a graph illustrating hMSC proliferation on apatite/gelatin-HPA cement;

FIG. 16 is a graph illustrating osteoinductivity of apatite/gelatin-HPA cement; and

FIG. 17 shows another graph illustrating osteoinductivity of apatite/gelatin-HPA cement.

### DETAILED DESCRIPTION OF THE PREFERRED EMBODIMENTS

The present invention provides a curable bone cement comprising a curable binder and a filler, wherein the cement (and/or the binder) is capable of curing without substantial evolution of heat. The cement may be capable of curing on exposure to a curing agent. Any or all of the components of the curable binder and of the curing agent may be pharmaceutically, clinically and/or veterinarily acceptable. They may be non-toxic to a patient in which they are used. They may be biocompatible.

The curable binder may comprise a polymeric species, or macromolecular species, and may also comprise either crosslinking moieties attached to the polymeric species or a crosslinking species mixed with the polymeric species. The polymeric species may be biocompatible. It may be non-toxic. It may for example be a glycosaminoglycan, a polysaccharide, a polycarboxylic acid, chondroitin, chondroitin sulfate, dermatan sulfate, heparan sulfate, heparin, proteoglycans, polyuronic acids (e.g. polypectate, polygalacturonic acid, polyglucuronic acid, pectin, colominic acid, alginate or some other polymeric species, and may be substituted. A suitable polymeric species is hyaluronan or hyaluronic acid, which may be substituted. The substituents may be the crosslinking moieties. The crosslinking moieties may comprise —C₆R′₄OR (i.e. phenol) groups which are capable of reacting in order to cure the cement. In the —C₆R′₄OR groups, R and each R′ may, independently, be hydrogen, an alkyl group, an aryl group or an acyl group, and R′ may also be OH, and each R′ is the same as or different to each other R′, provided that at least one R′, for example ortho to the OR group, is hydrogen, and wherein R and R′ are such that one —C₆R′₄OR group is capable of oxidatively coupling with another —C₆R′₄OR group. The other —C₆R′₄OR group may be attached to a different molecule of the polymeric species, so that the oxidative coupling crosslinks the polymeric species. The alkyl group may be a C1 to C12 or more straight chain alkyl group. It may be a C3 to C12 or more branched or cyclic alkyl group, or may have a mixture of alkyl and cycloalkyl portions (e.g. it may be cyclohexylmethyl). Suitable alkyl groups include methyl, ethyl, propyl etc. It will be understood that other substituents may be used, including alkenyl, alkynyl, aryl, heteroaryl groups etc. The nature of the groups R and R′ should not be such as to prevent oxidative coupling of —C₆R′₄OR groups. Thus for example excessively bulky substitutents, particularly the R′ groups which are on the ring, may inhibit or prevent coupling of the groups due to steric hindrance. Certain R′ groups may inhibit or prevent coupling due to electronic factors. At least some of the —C₆R′₄OR groups may be —C₆H₄OH groups, e.g. p-C₆H₄OH, or —C₆H₂(OH)₃, e.g. 3,4,5-trihydroxyphenyl groups. At least some of the phenol groups may be fused ring phenol groups e.g. a chromane structure bearing at least one phenolic OH group.

The binder may be generated by coupling the —C₆R′₄OR groups to a polymeric species (a polymer or an oligomer), optionally a biocompatible or non-toxic polymer or oligomer. The polymeric species may be a biopolymer. It may be a polysaccharide, a polyamine or a polypeptide, e.g. hyaluronic acid, gelatin or collagen. The coupling may comprise reacting the polymeric species with an aminofunctional phenolic species which comprises the —C₆R′₄OR group. Thus the amine group may be capable of coupling with a functional group (e.g. carboxylate, haloalkyl etc.) in the polymeric species. A suitable aminofunctional species may have formula H₂N-L-C₆R′₄OR, wherein R and R′ are as described above, and L is a linker group. L may be alkylene, arylene or some other suitable linker group e.g. methylene (—CH₂—), ethylene (—CH₂CH₂—), propylene (—CH₂CH₂CH₂—) etc. and may be straight chain, branched or cyclic. A suitable aminofunctional species may be tyramine (Tyr).

Alternatively or additionally, the coupling may comprise reacting the polymeric species with a non-aminofunctional phenolic species, such as a polyphenol. Suitable polyphenols include catechin, epicatechin, gallic acid and epigallocatechin gallate (EGCG). In this case, the phenol species may be conjugated to the polymer or oligomer by forming a conjugate of the polymer or oligomer with an acetal compound (e.g. a dialkyl acetal compound) to form an acetal-functional polymer or oligomer, and coupling the acetal-functional polymer or oligomer with the phenol species. For example, if the polymer is HA and the phenol species is EGCG, then EGCG may be coupled with a HA-acetal (e.g. HA-dialkylacetal) conjugate. This may be accomplished by conversion of the acetal functional group of the acetal-functional polymer or oligomer with an acid to generate an aldehyde functional group. The HA-dialkyl acetal may be formed by reaction of HA with an aminofunctional acetal (e.g. dialkylacetal), such as aminoacetaldehyde diethylacetal. This reaction may be conducted in aqueous solution under acidic conditions, commonly mildly acidic conditions (e.g. pH between about 4 and about 6), optionally in the presence of a condensation reagent such as N-hydroxysuccinimide and/or a carbodiimide. The reaction may be conducted at room temperature or at an elevated temperature, and may take from about 1 and about 24 hours, depending on the reagents, concentrations and temperature. The resulting HA-acetal conjugate may be purified by any of the well known methods, for example dialysis. The HA-acetal conjugate may then be hydrolysed using acid. It may for example be dissolved in water and the resulting solution hydrolysed by adjusting to pH below about 2 (e.g. about 1). This may be accomplished using a strong acid, e.g. a mineral acid such as hydrochloric acid, sulfuric acid or some other convenient acid. Addition of the phenol species (e.g. EGCG), optionally in solution (conveniently in a water miscible organic solvent such as DMSO, DMF etc.), to the resulting solution may result in production of the desired HA-phenol species conjugate. The latter reaction may be conducted at room temperature, or at some convenient elevated temperature that does not cause deterioration of the reagents or product. The reaction may be conducted under an intert atmosphere e.g. nitrogen, argon, carbon dioxide. It may take between about 1 and 48 hours, depending on the reagents, concentrations and temperature.

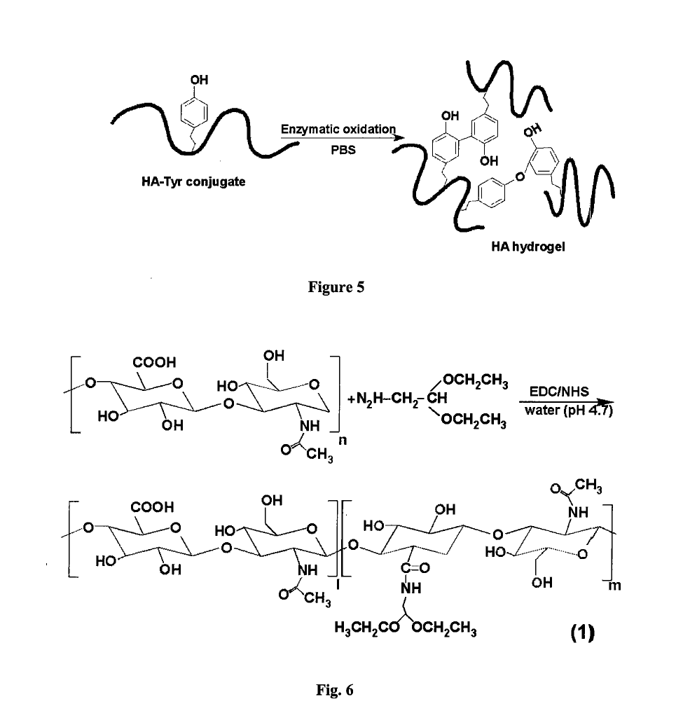

The structure of the binder may be backbone-linker-phenol group, where the backbone is derived from the polymeric species, and the phenol group is derived from the phenolic species. The binder may be made by coupling the linker to the polymeric species to form a backbone-linker combination and then coupling the phenol group to the backbone-linker combination, or it may be made by coupling the phenol group to the linker to provide a linker-phenol group combination (or the linker-phenol group combination may be provided from some other source, e.g. it may be available commercially, for example as tyramine) and coupling the linker-phenol group combination with the polymeric species. For example in the case described above, the aminofunctional acetal, or the corresponding aminofunctional aldehyde, may be coupled to EGCG to form an aminofunctional EGCG derivative, and the aminofunctional EGCG derivative may then be coupled to HA to form the HA-EGCG conjugate. The reaction conditions for coupling the aminofunctional EGCG derivative to HA may be similar to those used for coupling the aminofunctional acetal to HA as described above. The reaction conditions for coupling the aminofunctional acetal or aldehyde to EGCG may be similar to those used for coupling HA-dialkyl acetal to EGCG as described above. On curing the cement of the present invention, the backbone-linker-phenol group structure may be converted to a backbone-linker-crosslinked phenol group structure. A partial structure of the backbone-linker-crosslinked phenol group is shown in FIG. 5, however the cured binder of the present invention comprises filler particles distributed within the hydrogel structure shown in FIG. 5.

The binder may for example comprise a polysaccharide having phenolic groups attached thereto, optionally via a linker group (L, as described above), whereby the phenolic groups are capable of crosslinking the polysaccharide by an oxidative coupling. The binder may comprise a hyaluronic acid-tyramine (HA-Tyr) conjugate. Other suitable conjugates may be used, for example conjugates with tyramine, catechin, epicatechin, gallic acid or epigallocatechin gallate (EGCG), or mixtures of any two or more thereof. These may be conjugates with hyaluronic acid, or with some other polymer or oligomer.

Alternatively a separate crosslinking species may be mixed with the polymeric species such that the crosslinking species can crosslink the polymer on exposure to a catalyst without evolution of substantial heat. The crosslinking may occur through carbon atoms on an phenol group of the crosslinking species (e.g. through a carbon atom bearing a hydrogen atom before said crosslinking) and/or through an oxygen atom attached to a phenol group of the crosslinking species. A representative crosslinked structure that could be formed by the crosslinking is shown in FIG. 5.

The filler may comprise an inorganic filler, e.g. a mineral filler. It may be a reinforcing filler. It may be non-toxic, and may be biocompatible. It may be non-irritant to a patient treated with the bone cement. It may be for example silica, alumina, zirconia, talc, mica, an apatite or a mixture of any two or more of these. Other suitable fillers are well known to those skilled in the art. Examples of suitable apatite fillers include hydroxyapatite, carbonated apatite and mixtures thereof. The filler may be capable of reacting with the curable binder, or may be incapable of reacting therewith. The filler may have a mean particle size of between about 1 and about 500 microns, provided that the cement (having the filler particles therein) is capable of being injected through a syringe needle. The syringe needle may be between about 18 and 30 gauge. The mean particle size of the filler may be between about 1 and 200 microns, or between about 1 and 100, 1 and 50, 1 and 20, 1 and 10, 1 and 5, 10 and 200, 50 and 200, 100 and 200, 10 and 100, 10 and 50, 200 and 500, 300 and 500, 200 and 300, 100 and 300, 50 and 300 or 50 and 100 microns, for example about 1, 2, 3, 4, 5, 10, 15, 20, 25, 30, 35, 40, 45, 50, 60, 70, 80, 90, 100, 110, 120, 130, 140, 150, 160, 170, 180, 190, 200, 250, 300, 350, 400, 450 or 500 microns. The filler may have a narrow or broad particle size distribution. The filler may have a maximum particle size that is smaller than the internal diameter of the syringe needle (optionally less than 50% or the internal diameter to the syringe needle). The filler may be crystalline. It may be in the form of nanocrystals. It may comprise, or may consist essentially of, crystallites. It may have a mean (or maximum) crystallite size (or diameter) of less than about 1 micron, or less than about 200, 100 or 50 nm, or of about 10 to about 200 nm, or about 10 to 150, 10 to 100, 10 to 50, 50 to 200, 50 to 150, 50 to 100, 100 to 150 or 150 to 200 nm, e.g. about 1, 2, 3, 4, 5, 10, 15, 20, 25, 30, 35, 40, 45, 50, 60, 70, 80, 90, 100, 150, 200 nm. The filler may be apatite powder the crystallite size of which is typically less than 100 nm. The crystallites may agglomerate to form particles. It may have a mean (or maximum) agglomerate particle size of less than about 1 micron, or less than about 500, 200, 100 or 50 nm, or of about 10 to about 1000 nm, or about 10 to 500, 10 to 100, 10 to 50, 50 to 1000, 50 to 500, 50 to 100, 100 to 500 or 500 to 1000 nm, e.g. about 10, 20, 30, 40, 50, 60, 70, 80, 90, 100, 150, 200, 250, 300, 350, 400, 450, 500, 600, 700, 800, 900 or 1000 nm. Where the filler used is apatite powder, the typical crystallite size of which is less than 100 nm, it may agglomerate to form particles of several hundred microns. For example, it may agglomerate to form particles of about 250 to 500 microns, or 250 to 350, 350 to 500 or 300 to 400 microns, e.g. about 250, 300, 350, 400, 450 or 500 microns. The filler may also incorporate drug delivery particles. The filler incorporating drug delivery particles may have a mean (or maximum) particle size of up to about 500 microns, or up to about 400, 300, 200 or 100 microns, or about 100 to about 500 microns, or about 100 to 300, 300 to 500 or 200 to 400 microns, e.g. about 100, 150, 200, 250, 300, 350, 400, 450 or 500 microns.

A suitable filler for the present invention comprises nanocrystalline apatites (20 nm) having nanocrystalline hydroxyapatite (40 nm) and carbonated apatite (15 nm).

The curing reaction of the cement (i.e. of the curable binder) occurs without substantial evolution of heat. In the context of this specification this is taken to mean that the heat evolved when the cement is cured in the body of a patient may be insufficient to cause damage to surrounding tissue or to components of the curable cement (e.g. proteins that may be incorporated therein). The curing reaction may evolve sufficiently little heat when the cement is cured in the body of the patient (i.e. when it is cured at the body temperature of the patient) that the temperature of the curable cement during the curing reaction does not increase by more than about 5 Celsius degrees, or does not increase by more than about 4, 3, 2, 1 or 0.5 Celsius degrees. The curing reaction may occur at the body temperature of a patient into which it is injected. This temperature will depend on the nature of the patient. It may be between about 35 and about 45° C., or between about 35 and 40, 40 and 45, 37 and 43 or 36 and 39° C., e.g. at about 35, 36, 37, 38, 39, 40, 41, 42, 43, 44 or 45° C. At the curing temperature, the curable cement (when exposed to the curing agent to form the catalysed curable cement) may become solid in between about 10 seconds and about 30 minutes, or about 20 seconds and 1 minute or 10 seconds and 15 minutes, 10 seconds and 5 minutes, 10 seconds and 2 minutes, 10 seconds and 1 minute, 10 and 30 seconds, 10 and 20 seconds, 30 seconds and 30 minutes, 1 and 30 minutes, 5 and 30 minutes, 10 and 30 minutes, 15 and 30 minutes, 20 seconds and 5 minutes, 20 seconds and 1 minute, 1 and 10 minutes, 1 and 5 minutes or 30 seconds and 2 minutes, for example in about 10, 15, 20, 25, 30, 35, 40, 45, 50 or 55 seconds or about 1, 1.5, 2, 2.5, 3, 3.5, 4, 4.5, 5, 6, 7, 8, 9, 10, 15, 20, 25 or 30 minutes, or 35, 40, 45, 50, 55 or 60 minutes.

The curing reaction may be a free radical reaction. It may be initiated by free radicals generated by reaction of the oxidant, for example by reaction of a peroxide with a peroxidase enzyme. It may comprise formation of ether linkages between the aromatic rings on different molecules of the binder. It may comprise formation of direct carbon-carbon linkages between the aromatic rings on different molecules of the binder. The carbon-carbon linkages may be between carbon atoms on the ring that are ortho and/or para to an oxygen substituent on the ring. The curing may comprise formation of both ether linkages and carbon-carbon linkages as described above.

The inventors have found that the crystalline phase and crystallite size of Apatite/Gtn-HPA cement matches that of trabecular bone apatite, and that the apatite/Gtn-HPA cement described herein has a similar chemical composition to trabecular bone. The bone cement may support in vitro stem cell growth. It may support in vitro stem cell proliferation. It may be capable of inducing stem cells to express bone-like behavior. It may be suitable for healing and regeneration of bone defects (e.g. in spinal fusion, bone and joint defects, osteoporotic fractures, maxillofacial and revision surgery, vertebroplasty etc.). It may have high storage modulus. It may have high yield stress. It may have high compressive stiffness. The cured bone cement may have suitable physical properties for use as a bone repair material. It may be non-toxic to cells (in either the cured or the uncured state).

The bone cement may be used for repair of a bone of a patient. It may be used in maxillofacial surgery and osteochondral surgery on a patient. It may be used for healing bone defects and deficiencies in a patient. It may be used in spinal fusion, correction of bone and joint defects and osteoporotic fractures, revision surgery or vertebroplasty in a patient. The patient may be a vertebrate, e.g. a mammal, a bird, a fish or a reptile. It may be a human or non-human mammal. It may be for example a human, dog, cat, horse, cow, pig, elephant, llama, goat, sheep or some other type of mammal.

Curing of the curable binder, and of the curable bone cement, may be promoted by a curing agent. The curing agent may comprise an oxidant. The oxidant may be a mild oxidant so that curing of the cement may be accomplished without substantial evolution of heat. The curing agent may be a reagent for promoting (e.g. catalysing) the oxidative coupling of phenolic groups. The curing agent may comprise an enzyme, e.g. a peroxidase. It may comprise a peroxide. It may comprise a combination of a peroxide and an enzyme e.g. a peroxidase such as horse radish peroxidase (HRP). For example, the curing agent may comprise hydrogen peroxide and horse radish peroxidase.

The bone cement may additionally comprise one or more further component such as collagen, a silicate, a protein (e.g. growth factor) and platelets. The further components may serve to reinforce the cured bone cement, or may serve to promote healing of the bone into which the curable bone cement is injected or of surrounding tissue, or may serve to minimise damage or irritation to surrounding tissue or may serve some other purpose. The further component may be provided in a polymer-inorganic composite drug/protein/growth factor delivery particles in order to deliver healing agents. It may comprise controlled release delivery particles for delivering the healing agents to sites near or adjacent to the region where the cement is injected.

The bone cements of the present invention may be aqueous. They may comprise water. They may comprise an aqueous buffer solution. They may comprise PBS (phosphate buffered saline). They may comprise about 20 to about 80% by weight or weight/volume of water. They may comprise about 20 to 60, 20 to 40, 40 to 80, 60 to 80 or 40 to 60%, e.g. about 20, 25, 30, 35, 40, 45, 50, 55, 60, 65, 70, 75 or 80% by weight or weight/volume of water.

The bone cement (curable or catalysed) may be injectable. It may be in the form of a paste, or a slurry or some other viscous preparation. It may show rheology such that it is injectable using a syringe (e.g. between about 18 and 30 gauge), i.e. at relatively high shear it may be relatively non-viscous (mobile). It may show rheology such that, once injected into a bone, it will not readily flow out of place, i.e. at low shear it may be relatively viscous. It may display a yield stress, such that at shear stresses below the yield stress it does not flow.

The curable bone cement may be made by combining a solution of the curable binder with the filler, and optionally with one or more further component such as collagen, a silicate, a protein (e.g. growth factor) and platelets. The solution may be an aqueous solution. It may comprise additional components for example buffer materials. The solution may be prepared by dissolving the curable binder in a solvent, or may be prepared by combining a solution of a polysaccharide with a reagent, wherein the reagent comprises a crosslinking moiety, such that the polysaccharide reacts with the reagent to form the curable binder. The curable binder should have sufficient crosslinking moieties coupled thereto, or should have sufficient crosslinking species mixed therewith, that the curable cement, once cured to a solid cement, has an acceptable strength and/or hardness. The solid cement may have a wet compressive stiffness of at least about 0.5 MPa, or at least about 1, 2, 5, 10, 50, 100, 200, 300, 400, 500, 600, 700, 800, 900 or 1000 MPa. The wet compressive stiffness may be between about 0.5 MPa and 1 GPa, or between about 1 MPa and 1 GPa, 10 MPa and 1 GPa, 100 MPa and 1 GPa, 500 MPa and 1 GPa, 0.5 and 500 MPa, 0.5 and 100 MPa, 0.5 and 50 MPa, 0.5 and 20 MPa, 0.5 and 10 MPa, 0.5 and 5 MPa, 0.5 and 1 MPa, 1 and 500 MPa, 10 and 500 MPa, 100 and 500 MPa, 10 and 100 MPa or 10 and 50 MPa, and may have a wet compressive stiffness of about 0.5, 1, 1.5, 2, 2.5, 3, 3.5, 4, 4.5, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800 or 900 MPa or about 1 GPa. The crosslink density of the solid cement may be between about 1 and about 50 crosslinks per 100 monomer units of the polymeric species or between about 1 and 25, 1 and 10, 1 and 5, 5 and 50, 10 and 50, 25 and 50, 5 and 25 or 5 and 10 crosslinks per 100 monomer units, e.g. about 1, 2, 3, 4, 5, 10, 15, 20, 25, 30, 35, 40, 45 or 50 crosslinks per 100 monomer units. Thus for example if the curable binder comprises a HA-Tyr conjugate, the molar ratio of HA to Tyr (i.e. to sugar units of the HA) in making the conjugate may be between about 100:1 and 100:50 (based on the sugar units of HA). The solution of the curable binder may be between about 1 and about 10% w/v, or between about 1 and 5, 1 and 2, 2 and 10, 5 and 10, 1 and 3, 2 and 4 or 2 and 3%, for example about 1, 1.5, 2, 2.5, 3, 3.5, 4, 4.5 or 5%. The solution may be combined with the filler in a ratio of between about 1:5 and about 5:1, or between about 1:5 and 1:1, 1:1 and 5:1, 1:4 and 4:1, 1:3 and 3:1, 1:2 and 2:1 or 1:1.5 and 1.5:1, for example about 1:5, 1:4.5, 1:4, 1:3.5, 1:3, 1:2.5, 1:2, 1:1.5, 1:1, 1.5:1, 2:1, 2.5:1, 3:1, 3.5:1, 4:1, 4.5:1 or 5:1 on a w/w basis. The solution of the curable binder and the filler may be combined e.g. mixed, blended, homogenised, vortexed etc. to form the curable bone cement. If further components are included in the cement, they may be added after combining with the filler or before, or at the same time. It will be readily understood that the order of addition at this stage is not critical, and any convenient order may be employed. The further components may be added neat or in solution (e.g. aqueous solution), and if more than one further components are used, they may be added together or separately. For example the further component may be added to the combined curable binder and filler, or the curable binder may be combined with the combined filler and further component (optionally in solution). The ratio of filler to further component may depend on the nature of the filler and of the further component. The ratio may be for example between about 1:2 and about 100:1 on a w/w basis, or between about 1:2 and 50:1, 1:2 and 20:1, 1:2 and 10:1, 1:2 and 5:1, 1:2 and 2:1, 1:2 and 1:1, 1:1 and 100:1, 10:1 and 100:1, 50:1 and 100:1, 1:1 and 50:1, 1:1 and 20:1, 1:1 and 10:1, 1:1 and 5:1, 1:1 and 2:1, 5:1 and 50:1, 5:1 and 20:1 or 5:1 and 10:1, for example about 1:2, 1:1.5, 1:1, 1.5:1, 2:1, 2.5:1, 3:1, 4:1, 5:1, 6:1, 7:1, 8:1, 9:1, 10:1, 15:1, 20:1, 25:1, 30:1, 40:1, 50:1, 60:1, 70:1, 80:1, 90:1 or 100:1 or some other ratio.

In order to form a catalysed bone cement, the curable bone cement is exposed to the curing agent. The catalysed bone cement is curable. It may be curable without addition of further substances. It may be curable without heating. The curing agent may be combined with, e.g. mixed with, stirred with, shaken with, blended with, sonicated with or otherwise combined with the curable cement. The curing agent may be added in sufficient quantity that the bone cement cures at the temperature of use in the desired time. Temperatures and times for curing/setting have been described elsewhere in this specification. This quantity will depend on the nature of the curable cement and of the curing agent. As an example, if the curable cement comprises an HA-Tyr conjugate and the curing agent comprises HRP and hydrogen peroxide, the HRP may be added to the HA-Tyr at between about 0.01 and about 0.05 Units/mg (or between about 0.01 and 0.03, 0.01 and 0.02, 0.02 and 0.05, 0.03 and 0.05, 0.02 and 0.04 or 0.02 and 0.03, e.g. about 0.01, 0.015, 0.02, 0.025, 0.03, 0.035, 0.04, 0.045 or 0.05 Units/mg) and the hydrogen peroxide may be added at about 0.5 and 5 nmol/mg, or between about 0.5 and 2, 0.5 and 1, 1 and 5, 2 and 5, 1 and 3 or 0.8 and 1.2 nmol/mg, e.g. 0.5, 0.6, 0.7, 0.8, 0.9, 1, 1.1, 1.2, 1.3, 1.4, 1.5, 2, 2.5, 3, 3.5, 4, 4.5 or 5 nmol/mg. The HRP and the hydrogen peroxide may each be added in solution e.g. aqueous solution. They may be added together or separately. The concentration of HRP in the solution thereof may be between about 10 and about 100 U/ml (or between about 10 and 50, 10 and 20, 20 and 100, 50 and 100, 20 and 80, 15 and 30, 20 and 30 or 22 and 28 U/ml, e.g. about 10, 15, 20, 25, 30, 35, 40, 45, 50, 55, 60, 65, 70, 75, 80, 85, 90, 95 or 100 U/ml). The concentration of hydrogen peroxide in the solution thereof may be between about 1 and 10 mM, or between about 1 and 5, 1 and 2, 2 and 10, 5 and 10, 2 and 8, 3 and 7 or 4 and 6 mM, for example about 1, 2, 3, 4, 5, 6, 7, 8, 9 or 10 mM.

The curable bone cement may therefore be combined with the curing agent to form a catalysed bone cement. The cement may then be applied to the bone to be repaired, e.g. it may be injected into the bone or onto the bone or both. This should be accomplished before the curing reaction has proceeded to the point where the cement is no longer injectable. This will depend on the curing time, which is described elsewhere in this specification. It will be understood that commonly the curing reaction will be accelerated at elevated temperatures. Thus the catalysed bone cement may be prepared at relatively low temperatures (e.g. between about 10 and about 25° C. or 10 and 20, 10 and 15, 15 and 25, 20 and 25 or 15 and 20° C., for example about 10, 15, 20, 25° C. or ambient temperature), at which the curing rate is relatively slow, and may then be injected into a patient with a body temperature between about 35 and 45° C., as described earlier, at which the curing rate may be more rapid.

In one form, the present invention provides an injectable bone cement material comprising of hyaluronic acid-tyramine (HA-Tyr) conjugates and apatites. This injectable paste is capable of setting quickly via the formation of crosslinked network of HA in the presence of horseradish peroxidase (HRP) and hydrogen peroxide. The system shows no, or low, heat release during the formation of bone cements and no, or negligible, or acceptably low, tissue damage because the crosslinking reaction occurs by enzymatic oxidative reaction of the tyramine moiety in the HA-Tyr conjugates under mild conditions. This novel injectable HA-apatite-based bone cement is particularly well-suited for the healing of osteochondral defects as it contains mainly HA, collagen and apatites, all of which are native to the bone and joint regions.

HA is a glycosaminoglycan comprised of linear, unbranched, polyanionic disaccharide units. The disaccharide units consist of glucuronic acid N-acetyl glucosamine units joined alternately by beta-1,3 and beta-1,4 glycoside bonds. Tyramine is 4-(2-aminoethyl)phenol.

The inventors also have developed an injectable bone substitute material composed of apatite nanocrystals and gelatin-3-(4-hydroxyphenyl)propionic acid (Gtn-HPA) conjugates. In some formulations, collagen, silicates, agarose, proteins (e.g. growth factors) and/or platelets were also added. In addition, this material could be combined with the apatite/hyaluronic acid-tyramine (HA-Tyr) injectable bone cement described above to form a hybrid cement. The material would form an injectable paste when mixed with a solution containing horseradish peroxidase (HRP) and hydrogen peroxide (H₂O₂), and set within a short period to form a solid material by the cross-linking reaction of the Gtn-HPA conjugates. This technology allows for the regeneration of bone tissues at the defects by the injection of a mixture of apatite nanocrystals, Gtn-HPA conjugates, HRP, H₂O₂, collagen and/or growth factors. The main advantage of the bone cements described herein over conventional injectable bone cements is that the setting process involves very little heat release. This would prevent any damage of the surrounding tissues. The present injectable bone cement also provides the following additional benefits: (i) it does not require surgical implantation, (ii) it minimizes loss of biological activity of growth factors, and (iii) it provides for improved biocompatibility. The novel injectable bone cement described herein may be applied towards spinal fusion, repair of bone and joint defects, repair of osteoporotic fractures, maxillofacial and revision surgery, and vertebroplasty.

Thus another form of the curable bone cement of the present invention comprises a curable polymeric binder and a filler, wherein the cement is capable of curing without substantial evolution of heat on exposure to a curing agent, said binder comprising a reaction product of an aminofunctional polymer and an alkanoic acid bearing a phenolic group, said phenolic group in the binder being capable of reacting in order to cure the cement.

Where reference to a “reaction product” of two components is made, this should not be taken to indicate that the reaction product has necessarily been made by reacting the components, but should only be taken to indicate that the reaction product would have been formed if the components had been reacted. It should not be taken to exclude the same product made by some other route.

The reaction product of the aminofunctional polymer and the alkanoic acid may be one in which the amine functional groups (or at least some thereof) of the polymer have reacted with the carboxylic acid groups of the alkanoic acid. It may for example be an amidofunctional polymer, wherein the amide groups are amides of the alkanoic acid. It may be an ammonium salt of the polymer, wherein the ammonium salts are ammonium salts of the alkanoic acid. There may be some amide functionalities and some ammonium salt functionalities in the reaction product.

The phenolic group may be —C₆H₄OH. It may be 4-hydroxyphenyl. The 4-hydroxyphenyl group may be ring substituted (additionally to the 4-hydroxy group) or it may be unsubstituted (other than the 4-hydroxy group). The alkanoic acid may be terminally substituted with the phenolic group, e.g. a 4-hydroxyphenyl group. The longest straight chain of the alkanoic acid may be terminally substituted with the phenolic group, e.g. a 4-hydroxyphenyl group.

The alkanoic acid may be a C₂ to C₆ straight chain alkanoic acid. It may be ethyl, propyl, butyl, pentyl or hexyl. In other embodiments it may be a branched chain alkanoic acid, e.g. 2-ethylhexanoic acid or 2-methylpropionic acid.

The aminofunctional polymer may be an aminofunctional polysaccharide, a polyamine or a polypeptide, or may be a mixture of any two or all of these. The aminofunctional polymer may be a protein. The protein may be gelatine. The amine groups on the aminofunctional polymer may be primary amines. They may be secondary amines. They may be tertiary amines. They may be a mixture of any two or all of these.

The curing agent may comprise an oxidant. It may comprise a peroxide. It may comprise a hydroperoxide. It may comprise hydrogen peroxide. It may comprise a peroxidase enzyme and a peroxide, for example a hydroperoxide, more particularly hydrogen peroxide. It may comprise hydrogen peroxide and horse radish peroxidase. In the event that the curing agent comprises a peroxide and a peroxidase enzyme, these may not be mixed prior to being combined with the curable bone cement. They may be combined separately with the bone cement. The peroxide may be combined with the curable bone cement and the resulting mixture combined with the peroxidase enzyme, or the peroxidase enzyme may be combined with the curable bone cement and the resulting mixture combined with the peroxide.

The curable bone cement may be capable of curing to a solid in between about 10 seconds and about 30 minutes without substantial evolution of heat on exposure to the curing agent at the body temperature of a patient in which the cement is cured on exposure to the curing agent. Suitable cure times have been described earlier in this specification. In some embodiments the curing agent comprises two components, e.g. a peroxide and a peroxidase enzyme. In this event, these may be added separately to the curable bone cement. The above curing times should be understood to be from the time when the entire curing agent (i.e. both components) have been added to the curable bone cement. In effect therefore the curing times will be from the time of addition of the second component.

In the event that the curing agent comprises more than one component, they may be added sequentially to the curable bone cement. They may be added separately to the curable bone cement. They may be added simultaneously to the curable bone cement. They may be premixed and added together to the curable bone cement. They may be added separately and simultaneously (i.e. not premixed) to the curable bone cement. In the event that the curing agent comprises more than two components, some may be premixed and some may be added separately (either simultaneously or sequentially) or they may all be premixed and added together, or they may all be added separately (either simultaneously or sequentially).

The filler may comprise a mineral filler. It may comprise an apatite or a mixture of two or more apatites. Suitable fillers have been described earlier in this specification.

The curable bone cement may additionally comprise a second binder. The second binder may be a reaction product of a carboxylic acid functional polymer and an alkylamine bearing a second phenolic group. The second binder may be a binder as described in the first aspect of the invention (including any of the options and embodiments thereof). The said second phenolic group in the second binder should be capable of reacting in order to assist in the curing of the cement.

In this context, the term “assist” (and grammatically related terms such as “assisting”) indicates that molecules of the second binder react with each other and/or with molecules of the binder which is a reaction product of an aminofunctional polymer and an alkanoic acid bearing a phenolic group under the curing conditions to cure the bone cement. The cure may be viewed as a crosslinking reaction. In the presence of the second binder, the cure may be viewed as a co-crosslinking reaction or a copolymerisation reaction.

The carboxylic acid functional polymer of the second binder may be a biocompatible polymer. It may be a polysaccharide. The polysaccharide may be hyaluronic acid. In some cases a combination of different carboxylic acid functional polymers may be used. The alkylamine of the second binder may be a primary amine terminally substituted with a 4-hydroxyphenyl group. It may be a C1 to C6 primary amine terminally substituted with a 4-hydroxyphenyl group. It may be tyramine. Suitable second binders (in particular suitable amines and polymers) have been discussed earlier in conjunction with the first aspect of the invention (as the binder in that aspect).

The second binder may be capable of reacting in order to assist in the curing of the cement under the same conditions as are required for curing of the binder which comprises a reaction product of an aminofunctional polymer and an alkanoic acid bearing a phenolic group. This enables the second binder to cure with the binder so as to couple molecules of each in order to cure the curable bone cement. The cure conditions have been discussed earlier. In particular, suitable cure conditions include those pertaining in the body of a patient in the vicinity of a bone of said patient, as the bone cement may be used in at least partially repairing a bone in a patient.

The curable bone cement may additionally comprise at least one further component. The further component may be selected from the group consisting of collagen, a silicate, a protein and platelets. Suitable further components have been discussed earlier. It may also comprise water, or an aqueous solution e.g. an aqueous buffer solution.

The curable bone cement described herein may be combined with a curing agent, for example a peroxidase enzyme in combination with a peroxide, to form a catalysed bone cement. This may then be injected into a patient, in particular in the vicinity of a bone of said patient, in order to at least partially repair the bone. Commonly the catalysed bone cement will be in the form of an injectable paste. This enables the cement to be injected to a site in a patient where it is required, and to remain in place until cured.

The curable bone cement may be made by combining the curable polymeric binder and the filler. The process may also comprise preparing the curable polymeric binder. This step may comprise reacting an aminofunctional polymer and an alkanoic acid bearing a phenolic group. In the event that the polymer is a protein, this may be under conditions that do not denature the protein. It may be catalysed by a catalyst that does not denature the protein. It may be conducted at a temperature at which the protein does not denature.

The curable polymeric binder may be dissolved in a solvent prior to said combining. The solvent may be an aqueous solvent. It may be an aqueous buffer solution. It may for example be PBS solution. It may be a biocompatible solvent. It may be a non-toxic solvent. The concentration of the polymeric binder in the solvent may be about 20 to about 100 mg/ml, or about 20 to 50, 50 to 100 or 40 to 60 mg/ml, e.g. about 20, 25, 30, 35, 40, 45, 50, 55, 60, 65, 70, 75, 80, 85, 90, 95 or 100 mg/ml.

The filler may be present at up to about 20 times the amount of binder by weight, or up to about 15, 10, 5, 2 or 1 times the amount of binder, or about 1 to 20, 5 to 20, 10 to 20, 1 to 15, 1 to 10, 1 to 5, 5 to 15, 5 to 10 or 10 to 15 times, e.g. about 1, 2, 3, 4, 5, 6, 7, 8, 9, 10, 11, 12, 13, 14, 15, 16, 17, 18, 19 or 20 times.

The second binder, if used, may be combined with the binder comprising a reaction product of an aminofunctional polymer and an alkanoic acid bearing a phenolic group. In this case the total concentration of binder plus second binder may be as described above (about 20 to about 100 mg/ml). The ratio of the binder to the second binder may be about 2:1 to about 1:2 on a weight basis, or 1.5:1 to 1:1.5 or 2:1 to 1:1 or 1:1 to 1:2, e.g. about 2:1, 1.5:1, 1.2:1, 1:1, 1:1.2, 1:1.5 or 1:2.

The process for making the durable bone cement may comprise making the filler. In the event that the filler is an apatite, e.g. hydroxyapatite or carbonated apatite (or a mixture of these), this may be accomplished by combining calcium nitrate, ammonium phosphate and ammonium carbonate, or suitable similar salts. The process may be such that the filler forms as nanocrystals.

The curable bone cement may be cured by exposing the curable bone cement to a curing agent to form a catalysed bone cement and curing the catalysed bone cement without substantial evolution of heat. The step of curing the catalysed bone cement may simply comprise allowing the cement to cure. It may comprise allowing it to sit at a suitable temperature for cure. In the present context, “without substantial evolution of heat” should be taken to indicate that the heat evolved is insufficient to cause damage to surrounding tissue, or to bone or to the cement itself when cured adjacent to bone in a patient. As discussed elsewhere, in cases where the curing agent comprises separate components (e.g. a peroxidase enzyme and a peroxide) these may be added discretely to the curable cement, or at least one may be added discretely from at least one other component.

The curing agent has been described above. Commonly in the event that the curing agent comprises a peroxidase enzyme and a peroxide, the ratio of enzyme to binding agent is about 1 to about 5 units of enzyme to 1 g, or about 1 to 3, 3 to 5 or 2 to 4 units per gram, or about 1, 2, 3, 4 or 5 units per gram. The concentration of peroxide used may be between about 1 to about 50 mM, or about 1 to 25, 1 to 10, 5 to 50, 10 to 50, 25 to 50, 5 to 25, 10 to 25 or 10 to 40, e.g. about 1, 2, 3, 4, 5, 10, 15, 20, 25, 30, 35, 40, 45 or 50 mM. Where the curing agent comprises a peroxide and a peroxidase enzyme, these may be added discretely, in sequence, just before curing of the curable bone cement is desired (e.g. within minutes). The peroxide may be added before the enzyme or it may be added after the enzyme. Following addition of the first component of the curing agent to the bone cement, the resulting combination may optionally be mixed prior to adding the second component of the curing agent in order to homogenise said first component in the curable bone cement. Following addition of the second component of the curing agent, the resulting catalysed bone cement may be mixed prior to, optionally also during, curing. Where the curing agent comprises a peroxide and horse radish peroxidase, the peroxide may be added just before adding the horse radish peroxidise. The peroxide and horse radish peroxidase may be added separately, in sequence, just before curing is desired. Upon adding the horse radish peroxidase, curing typically occurs within minutes. Thus peroxide degradation is not a problem as the catalysed bone cement typically cures before a significant degree of degradation could occur. Concentrations of peroxide commonly persist in the catalysed bone cement and the final peroxide concentration may affect the crosslinking of the catalysed bone cement. The initial concentration of peroxide used may typically be a solution of about 1 to 5% by weight or weight/volume.

The bone cement described herein may be used to at least partially repair a bone in a patient. Thus the curing method may additionally comprise the step of injecting the bone cement into a patient before the step of curing the catalysed bone cement. In particular it may comprise injecting the bone cement into the patient in the vicinity of a region of damage (e.g. a fracture, break, hairline fracture, compression fracture, indentation etc.) of a bone of the patient. The amount of cement injected may be sufficient to improve the strength of the bone. It may be sufficient to at least partially repair the bone. Clearly the actual amount will vary depending on the precise nature (strength etc.) of the cement, the nature of the fracture and the size of the bone to be repaired. The bone cement may be capable of bonding to the bone in vivo. It may be capable of bonding by adhering chemically to the bone. It may be capable of bonding by means of at least partial penetration and/or intercalation into the bone.

The curable bone cement of the invention may comprise added collagen, silicates, and/or proteins such as growth factors and platelets. The cement forms an injectable paste (i.e. a catalysed bone cement) when mixed with a solution containing HRP and hydrogen peroxide. It sets within a short time to form a solid material by the crosslinking of the HA-Tyr conjugates. The main advantage of the bone cement of the present invention over traditional injectable bone cements is that the setting process does not release heat, which would damage the surrounding tissues. Evolved heat may also damage components of the cement, for example included growth factor. During cure of the presently described bone cements, the heat evolved (as measured by DSC) may be less than about 10 J/g, or less than about 5 or 2 J/g, or may be about 1 to about 10 J/g, or about 3 to 10, 5 to 10, 1 to 5, 1 to 2, 2 to 5, 4 to 7 or 4 to 5 J/g, e.g. about 1, 1.5, 2, 2.5, 3, 3.5, 4, 4.5, 5, 5.5, 6, 6.5, 7, 7.5, 8, 8.5, 9, 9.5 or 10 J/g.

The storage modulus of the cured bone cement (G′) may be about 100 to about 250 kPa, or about 100 to 1200, 100 to 150, 150 to 250, 200 to 250 or 150 to 250 kPa, for example about 100, 110, 120, 130, 140, 150, 160, 170, 180, 190, 200, 210, 220, 230, 240 or 250 kPa. It may have a yield stress (σ_{y}) of about 10 to about 40 kPa, or about 10 to 30, to 20, 20 to 40, 30 to 40, 25 to 35 or 30 to 35 kPa, e.g. about 10, 15, 20, 25, 30, 35 or 40 kPa. It may have a compressive stiffness (E_{c}) of about 10 to about 200 kPa, or about 50 to 200, 100 to 200, 150 to 200, 100 to 150 or 130 to 150, e.g. about 10, 20, 30, 40, 50, 60, 70, 80, 90, 100, 105, 110, 115, 120, 125, 130, 135, 140, 145, 150, 155, 160, 165, 170, 175, 180, 185, 190, 195 or 200 kPa. It may have a swelling ratio of about 3 to about 8, or about 3 to 6, 3 to 4, 4 to 8, 5 to 8 or 4 to 6, e.g. about 3, 3.5, 4, 4.5, 5, 5.5, 6, 6.5, 7, 7.5 or 8. The swelling ratio is measured by swelling in water. A suitable swelling ratio measurement procedure is outlined under the sub-heading “Testing” in Example 2 in this specification. In a particular embodiment, the storage modulus is 225 kPa, yield stress is 31 kPa and compressive stiffness is 144 kPa.

The components and ratios thereof of the curable cement (e.g. one or more of the nature, crystallinity, particle size and shape, surface chemistry of the filler, chain length, functional group density, polymer architecture, chemical nature of the binder, ratios of filler to binder, proportion and nature of other components, particularly solvents) may be such as to provide one or more of the above properties. The conditions of cure (e.g. one or more of the proportion of curing agent to binder, proportion of peroxide to enzyme in curing agent, temperature etc.) may be such as to provide one or more of the above properties.

The cement of the present invention provides many benefits: (i) it does not require surgical implantation, (ii) it prevents tissue damage, (iii) it suffers less loss in biological activity for growth factors, and (iv) it provides for improved biocompatibility.

From the standpoint that the tissue surrounding the bone is mainly composed of HA and collagen, a bone cement according to the present invention, made using HA-Tyr conjugate with collagen, possesses the advantage that it enables the crystallization of apatite in the HA-collagen matrix without tissue damage. While many bone scaffolds containing HA and collagen have been reported, this bone cement is more versatile as it is possible, using this cement, to regenerate the bone tissue by a simple injection, without damaging surrounding tissue. The bone cement is also particularly well-suited to the healing of osteochondral defects as it contains mainly HA, collagen and apatites, all of which are native to the bone and joint regions. The bone cement may be especially suitable for use at the bone-joint interface as it primarily contains HA and apatites, which are the major constituents of cartilage and bone, respectively. It can be used as a graded composite structure for healing defects at this location.

Animal studies on mice have indicated that a bone cement according to the present invention was non-toxic and biocompatible, and set readily in vivo. In addition, the material also appeared to be osteoinductive as positive alkaline phosphatase staining results were obtained on the extracted samples 5 weeks post-injection.

### EXAMPLES

### Example 1

### Materials and Methods

Hydroxyapatite (HAP) and carbonated apatite (CAP) were synthesized from calcium nitrate, ammonium phosphate and ammonium carbonate by base precipitation. Collagen was extracted from rats, and dissolved in 0.05 M phosphoric acid at a concentration of 40 mg/ml. Four different formulations of injectable pastes were examined:

1. HA-Tyr solution only (control)

2. HA-Tyr solution and apatite powders

3. HA-Tyr solution and apatite powders, and collagen solution

4. HA-Tyr solution, and pre-mixed collagen-apatite solution

HA-apatite-based bone cements, both with and without collagen, set in mice by injection of the paste mixture of HA-Tyr, apatite, HRP and hydrogen peroxide. For the sample without collagen, HA-Tyr (25 mg) was dissolved in 1 ml of PBS (phosphate buffer solution). To this solution, 600 mg of apatite powder was added, followed by vortexing thoroughly. Freshly prepared 25 μl of HRP (25 U/ml) and 5 μl of hydrogen peroxide 0.14 mol/L) solutions were added to the paste of HA-Tyr as curing agent for the enzymatic oxidative coupling reaction. The paste was then injected subcutaneously through an 18-gauge needle into the Swiss albino mice where it set into a solid cement within 30 seconds from the time of addition of HRP and hydrogen peroxide. For the sample with collagen, we prepared two different paste solutions: (i) the paste solution of HA-Tyr and apatite containing 0.5 ml of collagen, and (ii) HA-Tyr solution containing 1 ml of pre-mixed solution of collagen and apatite.

5 weeks post-injection, the mice were sacrificed and the injected cement was removed for cryosectioning and histological analysis. The slides were immunostained using hematoxylin and eosin (H and E), alkaline phosphatase and nuclear fast red (ALP and NFR), and Von Kossa and nuclear fast red (VK and NFR) solutions.

### Results and Discussion

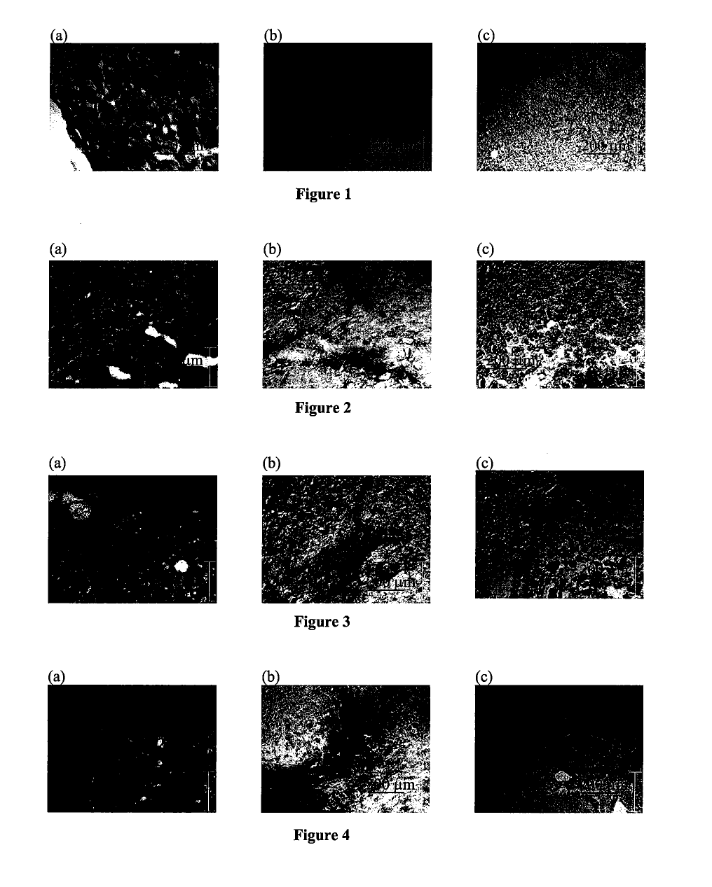

After 5 weeks post-injection, the following results were obtained. H and E staining showed that there was healthy cell proliferation, blood supply and tissue ingrowth with no necrosis for all samples (FIGS. 1(a), 2(a), 3(a) and 4(a)). (H and E is Hematoxylin and Eosin stain for histological tissue sections. Cell nuclei will be stained blue, with some metachromasia. Cell cytoplasm will be stained various shades of pink, identifying different tissue components. ALP is Alkaline Phosphatase Chromogen stain for histological sections (also known as BCIP/NBT; BCIP: 5-bromo-4-chloro-3-indolyl phosphate, NBT: p-nitroblue tetrazolium chloride). Areas with alkaline phosphatase activity will be stained a deep purple. Alkaline phosphatases are a group of enzymes found primarily the liver (isoenzyme ALP-1) and bone (isoenzyme ALP-2). NFR is Nuclear Fast Red stain, a counterstain for histological sections. Cell nuclei will be stained red and cell cytoplasm will be stained pink. VK is Von Kossa staining of histological sections for calcium. This technique is for demonstrating deposits of calcium or calcium salt, so it is not specific for the calcium ion itself. In this method, tissue sections are treated with a silver nitrate solution and the silver is deposited by replacing the calcium reduced by the strong light, and results in a black or brown-black stain in areas with calcium salts.) Compared to the control (FIG. 1(b)), the incorporation of apatites into the material formulation resulted in positive ALP staining, where areas of osteoblast activity were stained dark purple (FIGS. 2(b), 3(b) and 4(b)). Positive VK staining (dark brown) was also observed in the samples containing apatites (FIGS. 2(c), 3(c) and 4(c), which could be due to the calcium present in the apatites or released through osteoblast activity. This indicated that our materials were non-toxic and biocompatible. In addition, the apatite-containing formulations also appeared to be osteoinductive since ALP activity was observed after injection into an ectopic region.

### Conclusions

The inventors have synthesized bone cement materials that are injectable and fast-setting in vivo with no heat release or surrounding tissue damage. A simple and non-toxic injectable in situ bone cement system was achieved using an enzymatic oxidative coupling reaction. The biocompatibility and convenience of application of this injectable bone cement system would be highly advantageous to the healing and regeneration of bone defects.

Preliminary in vivo studies confirmed that the HA-apatite-based materials were non-toxic and biocompatible, and likely to be osteoinductive. These bone cements contain primarily hyaluronic acid and apatites, both of which are naturally abundant in the bone-joint area. These characteristics would make the materials particularly well-suited for the healing of defects in the osteochondral region, and for use in spinal fusion, bone and joint defects, osteoporotic fractures, maxillofacial and revision surgery, and vertebroplasty.

### Synthesis of hyaluronic acid-aminoacetylaldehyde diethylacetal Conjugate (1)

The conjugate (1) was synthesized by following a general protocol, which is shown in FIG. 6. HA (1 g, 2.5 mmol) was dissolved in 100 ml of distilled water. To this solution aminoacetaldehyde diethylacetal (1.2 g, 9 mmol) was added. The pH of the reaction mixture was adjusted to 4.7 by the addition of 0.1M HCl. N-hydroxysuccinimide (0.34 g, 3.0 mmol) and 1-ethyl-3-[3-(dimethylamino)propyl]carbodiimide hydrochloride (EDC) (0.575 g, 3.0 mmol) were added to the solution. After mixing, the pH of the reaction was maintained at 4.7. The solution was kept at room temperature for 24 h under gentle stirring. The mixture was subjected to purification by dialysis (molecular weight cut off 1000).

### Synthesis of hyaluronic acid-epigallocatechin gallate (HA-EGCG) Conjugate

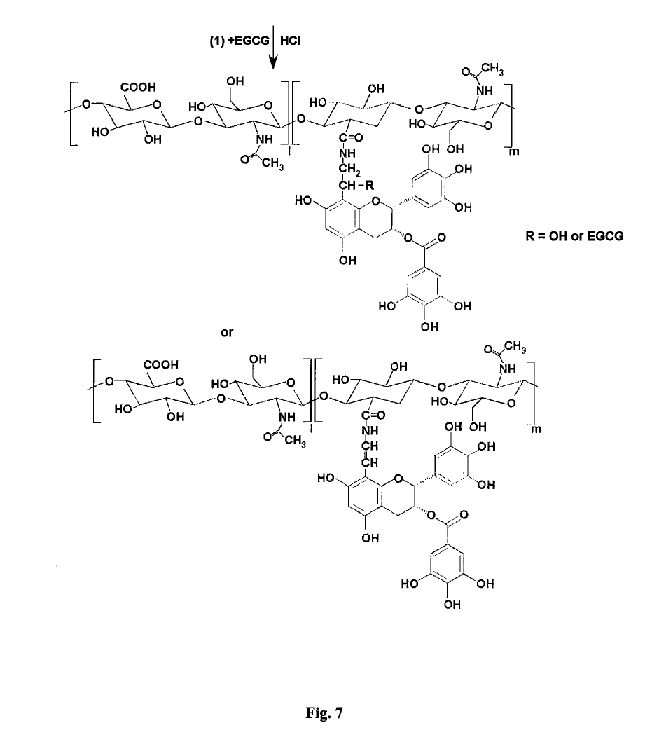

HA-EGCG conjugate was synthesized by the protocol shown in FIG. 7. 1 g of conjugate (1) was dissolved in 60 ml of distilled water. Then the pH of the solution was adjusted to 1 by adding HCl solution. To this solution 5 ml of EGCG solution dissolved in DMSO (0.2 g/ml) was added. The solution was kept at room temperature under nitrogen for 24 h under gentle stirring. The mixture was subjected to purification by dialysis (molecular weight cut off=1000).

### Example 2

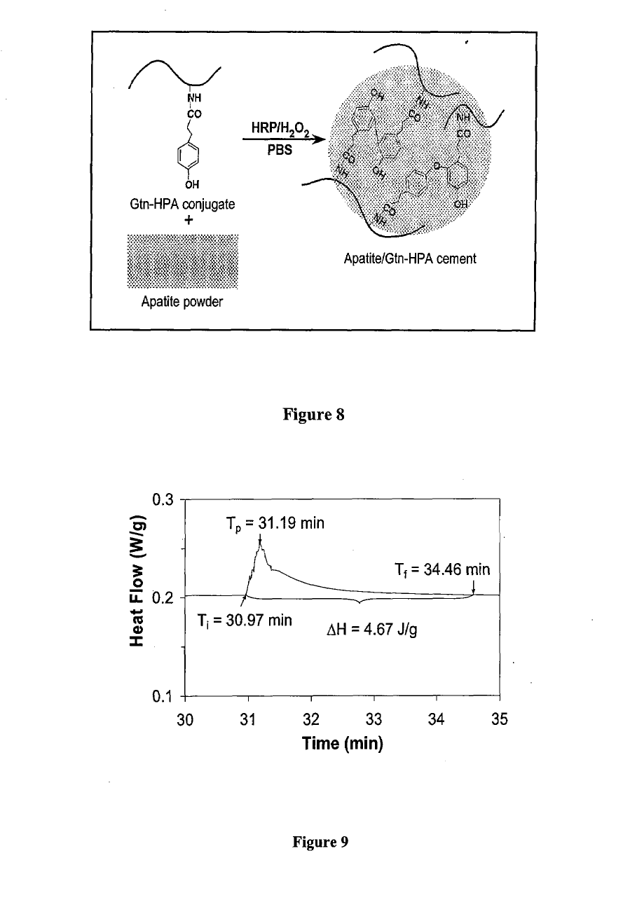

An injectable bone cement composed of apatite nanocrystals and gelatin-3-(4-hydroxyphenyl)propionic acid (Gtn-HPA) conjugates has been developed. This bone cement was formed using the oxidative coupling of HPA moieties catalyzed by hydrogen peroxide (H₂O₂) and horseradish peroxidase (HRP) (FIG. 8). The bone cement set within 5 min after H₂O₂ and HRP were added to the apatite/Gtn-HPA paste. This enzyme-mediated setting of our bone cement resulted in much less heat release (ΔH=−4.67 J/g) as compared to conventional bone cements. The mechanical strength of the apatite/Gtn-HPA cement was tuned by varying the apatite loading and H₂O₂ concentration. The swelling ratios of the cements with different H₂O₂ concentrations were measured to study the effect of H₂O₂ on crosslinking. A good correlation was observed between mechanical strength and swelling ratio; the mechanical strength of the cement increased with decreasing swelling ratio. The storage modulus (G′), yield stress (σ_{y}), and compressive stiffness (E_{c}) of the cured bone cement were optimized at G′=230 kPa, σ_{y}=31 kPa and E_{c}=144 kPa, respectively, when the cement was formed with 0.4 g/ml of apatite, 0.15 units/ml of HRP and 16.5 mM of H₂O₂.

### Introduction

Many clinical procedures such as maxillofacial surgery and osteochondral surgery require the use of bone cements to fill bone defects and deficiencies. Otherwise, the bone defects and deficiencies would not heal properly, preventing the return of normal function. Various synthetic bone substitutes have been developed for this purpose, some of which are produced in an injectable form, so as to enable minimally invasive surgery.

The main uses of injectable bone substitutes include spinal fusion, bone and joint defects, osteoporotic fractures, revision surgery, and vertebroplasty.

Described herein is an injectable bone substitute material based on apatite nanocrystals and Gtn-HPA conjugates. The material can form an injectable paste when mixed with a solution containing HRP and H₂O₂, and set within a short period to form a solid material by the cross-linking reaction of the Gtn-HPA conjugates.

### Materials and Methods

Hydroxyapatite (HAP) and carbonated apatite (CAP) were synthesized from calcium nitrate, ammonium phosphate and ammonium carbonate by base precipitation. 1-Ethyl-3-(3-dimethylaminopropyl)-carbodiimide hydrochloride (EDC.HCl), 3,4-hydroxyphenylpropionic acid (HPA) and collagenase were all purchased from Sigma-Aldrich. N-Hydroxysuccinimide (NHS) from Aldrich, hydrogen peroxide (H₂O₂) from Lancaster, while horseradish peroxidase (HRP, 100 units/mg) and gelatin were from Wako Pure Chemical Industries. Gtn-HPA was synthesized according to the methods described below.

### Synthesis of Gtn-HPA

The Gtn-HPA conjugates were prepared by a general carbodiimide/active ester-mediated coupling reaction in distilled water. HPA (3.32 g, 20 mmol), NHS (3.2 g, 27.8 mmol) and EDC.HCl (3.82 g, 20 mmol) were dissolved in 250 ml of mixture of distilled water and DMF (3:2). The reaction was stirred at room temperature for 5 hr, and the pH of the mixture was maintained at 4.7. Then, 150 ml of Gtn aqueous solution (6.25 wt. %) was added to the reaction mixture and stirred over night at room temperature at pH 4.7. The solution was transferred to dialysis tubes with molecular cut-off of 1000 Da. The tubes were dialyzed against 100 mM sodium chloride solution for 2 days, a mixture of distilled water and ethanol (3:1) for 1 day and distilled water for 1 day, successively. The purified solution was lyophilized to obtain the Gtn-HPA. The yield was 78-83%. The degree of HPA substitution to the amino groups of Gelatin (the number of phenol molecules per 100 amino acid residues of Gtn) was determined by the conventional 2,4,6-trinitrobenzene sulfonic acid (TNBS) method. See S. L. Synder, P. Z. Sobocinski, An improved 2,4,6-trinitrobenzenesulfonic acid method for the determination of amines, Analytical Biochemistry, 1975, 64, 284-288, which is herein incorporated by reference.

### Formation and Cure of Bone Cement

Gtn-HPA was dissolved in phosphate buffered saline (PBS) to form a 50 mg/ml solution, and mixed with various amounts of apatite nanocrystals. Gelation of the cement was induced by the addition of 0.15 units/ml of HRP and various concentrations of H₂O₂.

### Testing

Rheological and compression tests were performed on samples with a fixed HRP concentration of 0.15 units/ml to determine the optimal H₂O₂ concentration and apatite loading to produce the highest G′, σ_{y} and E_{c}. To measure G′, 200 μl of apatite/Gtn-HPA sample containing HRP and H₂O₂ were tested with a rheometer using a smooth parallel plate system with a diameter of 35 mm and a gap distance of 0.025 mm. To obtain σ_{y} and E_{c}, cylindrical samples of fully set apatite/Gtn-HPA cements (1 cm diameter×0.5 cm height) were subjected to compression tests.

To determine the swelling ratio of apatite/Gtn-HPA cements with different mechanical strengths, apatite/Gtn-HPA cements were prepared with 0.4 g/ml of apatite, 0.15 units/ml of HRP and various concentrations of H₂O₂. The pastes were allowed to set completely at 37° C. for 1 h, and were immersed in PBS at 37° C. for 3 days. The swollen cements were then gently blotted dry with Kimwipe™ and weighed to obtain the swollen weight. The cements were then frozen at −20° C., and lyophilized to obtain the dry weight. The swelling ratio was then obtained for each sample by calculating the ratio of swollen weight to dry weight.

### Results and Discussion

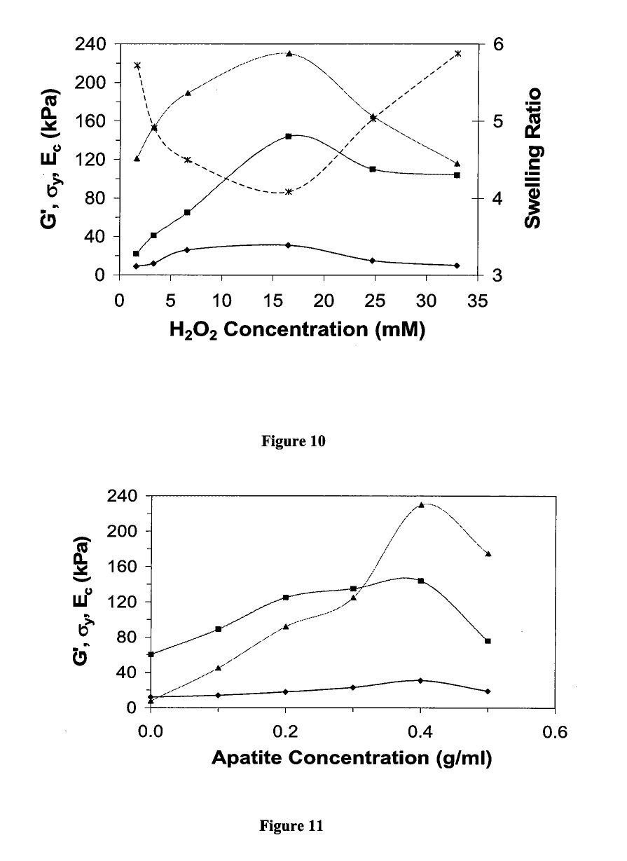

The bone cement set within 5 min after H₂O₂ and HRP were added to the apatite/Gtn-HPA paste. Differential scanning calorimetry (DSC) illustrated that this enzyme-mediated setting of bone cement involved very little heat release (ΔH=−4.67 J/g) (FIG. 9) as compared to conventional bone cements (traditional PMMA-based bone cements typically release about −70 to −90 J/g). The mechanical strength of the apatite/Gtn-HPA cement was tuned by varying the apatite loading and H₂O₂ concentration. The swelling ratios of the cements with different H₂O₂ concentrations were measured to study the effect of H₂O₂ on crosslinking. FIG. 10 shows that the mechanical strength of the cement increased with decreasing swelling ratio. The storage modulus (G′), yield stress (σ_{y}), and compressive stiffness (E_{c}) of the bone cement were optimized at G′=230 kPa, σ_{y}=31 kPa and E_{c}=144 kPa when the cement was formed with 0.4 g/ml of apatite, 0.15 units/ml of HRP and 16.5 mM of H₂O₂ (FIG. 11).

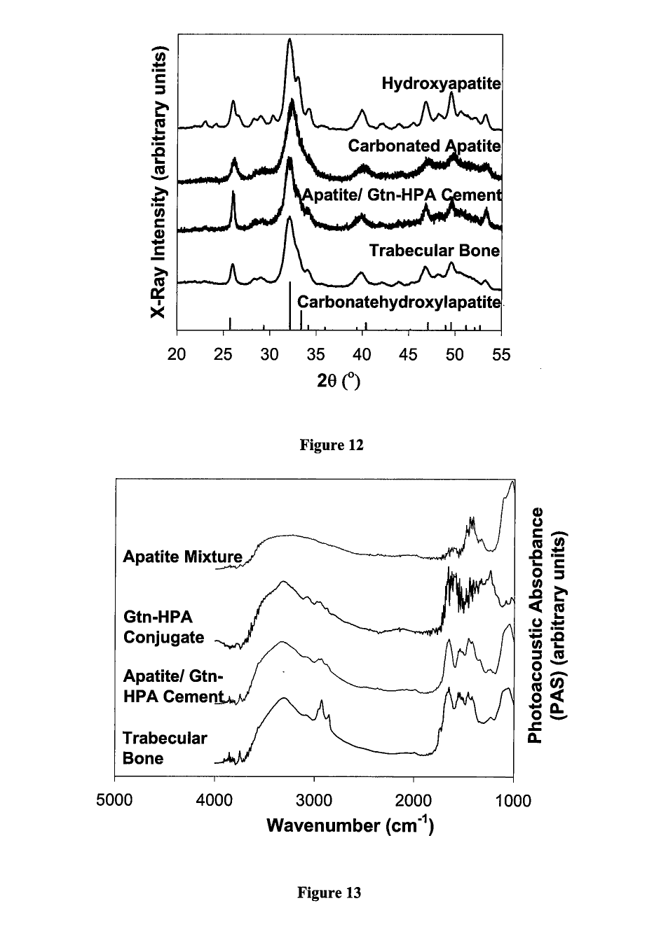

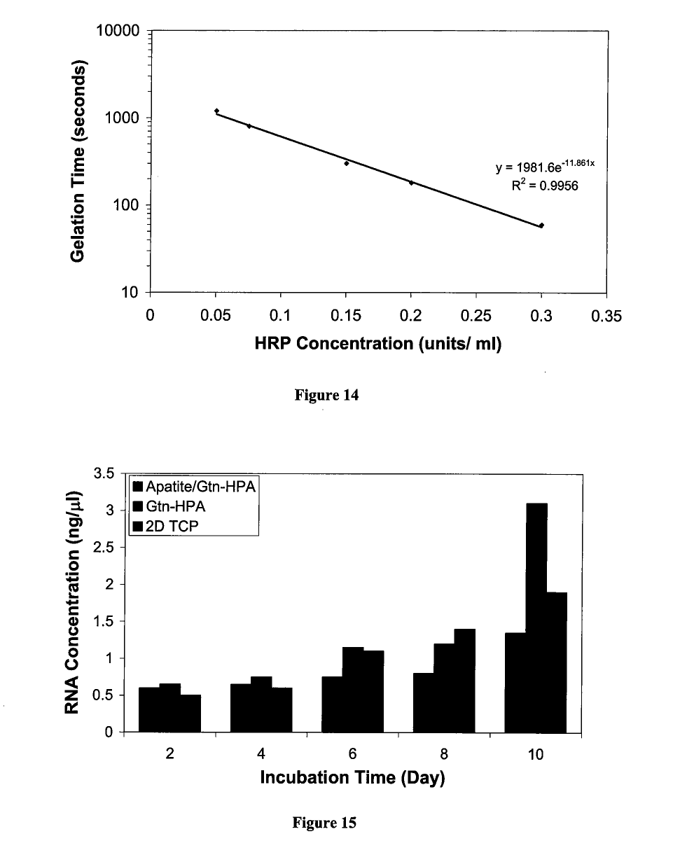

FIGS. 12 and 13 show, respectively, the physical and chemical similarities between the cured bone cement described in this example and trabecular bone. The cure rate (as reflected by gel time) of the curable bone cement could be adjusted readily by varying the amount of HRP added for cure. This is illustrated in FIG. 14, which shows a decrease in gel time with increased concentration of HRP added. Gelation time was about 300 seconds when the optimized bone cement was formed with 0.4 g/ml of apatite, 16.5 mM of H₂O₂ and 0.15 units/ml of HRP. This allows sufficient time for mixing and injection of cement, without causing much diffusion of gel precursors.

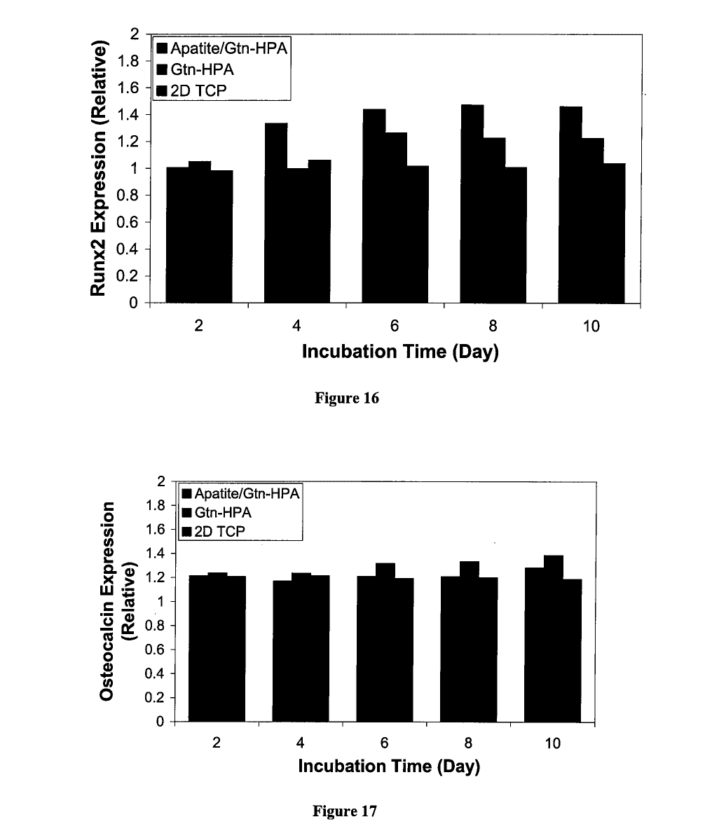

FIGS. 15 to 17 demonstrate the utility of the bone cement. FIG. 15 illustrates the growth of stem cells (hMSCs) on the bone cement, and demonstrates that they are able to survive and proliferate on apatite/Gtn-HPA cement to an extent comparable to 2D tissue culture plastic. FIG. 16 illustrates the osteoactivity of the bone cement. Runx2 expression is associated with osteoblast differentiation. Thus levels of Runx2 expressed by hMSCs growing on apatite/Gtn-HPA cement were measured. Runx2 expression increased with time for both apatite/Gtn-HPA and Gtn-HPA. Levels were higher in apatite/Gtn-HPA due to both substrate stiffness and bioactive apatite. Gtn-HPA showed some increase due to the effect of substrate stiffness. FIG. 17 also illustrates the osteoactivity of the bone cement. Osteocalcin is secreted by osteoblasts and regulates bone mineralization. Thus levels of osteocalcin secreted by hMSCs growing on apatite/Gtn-HPA were measured. Osteocalcin level increased with time for both apatite/Gtn-HPA and Gtn-HPA. It is thought that the slower increase for apatite/Gtn-HPA may be due to negative feedback from apatite.

### Conclusions

The inventors have synthesized bone cement materials that are injectable and fast-setting in vivo with little heat release that would not damage the surrounding tissue. A simple, non-toxic and injectable bone cement system was achieved using an enzymatic oxidative coupling reaction. The biocompatibility and convenience of applying this injectable bone cement system would be very beneficial towards healing and regeneration of bone defects. The bone cement is well-suited for the healing of defects in the osteochondral region, and for use in spinal fusion, bone and joint defects, osteoporotic fractures, maxillofacial and revision surgery, and vertebroplasty. The composition of apatite/Gtn-HPA cement was optimised to obtain high storage modulus, yield stress and compressive stiffness. In vitro studies confirmed that the apatite/Gtn-HPA cement is non-toxic to cells. It was shown that the bone cement can support in vitro stem cell growth and proliferation as well as tissue culture plastic. It can also induce stem cells to express bone-like behavior (as demonstrated by increased Runx2 and Osteocalcin).

1. A curable bone cement comprising a curable polymeric binder and a filler, wherein the cement is capable of curing without substantial evolution of heat on exposure to a curing agent, said binder comprising a reaction product of:
an aminofunctional polymer, and
an alkanoic acid bearing a phenolic group,
said phenolic group in the binder being capable of reacting in order to cure the cement.

2. The curable bone cement of claim 1 wherein the alkanoic acid is terminally substituted with a 4-hydroxyphenyl group.

3. The curable bone cement of claim 1 wherein the aminofunctional polymer is a protein.

4. The curable bone cement of claim 3 wherein the protein is gelatine.

5. The curable bone cement of claim 1 wherein the curing agent comprises a peroxidase enzyme and a peroxide.

6. The curable bone cement of claim 1 wherein the filler comprises an apatite or a mixture of two or more apatites.

7. The curable bone cement of claim 1 additionally comprising a second binder, said second binder being a reaction product of:
a carboxylic acid functional polymer, and
an alkylamine bearing a second phenolic group
said second phenolic group in the second binder being capable of reacting in order to assist in the curing of the cement.

8. The curable bone cement of claim 7 wherein the carboxylic acid functional polymer is hyaluronic acid.

9. The curable bone cement of claim 7 wherein the alkylamine is a primary amine terminally substituted with a 4-hydroxyphenyl group.

10. The curable bone cement of claim 7 wherein the second binder is capable of reacting in order to assist in the curing of the cement under the same conditions as are required for curing of the binder defined in claim 1.

11. The curable bone cement of claim 1 additionally comprising at least one further component selected from the group consisting of collagen, a silicate, a protein and platelets.

12. The curable bone cement of claim 11 wherein the protein is a growth factor.

13. A catalysed bone cement comprising the curable bone cement of claim 1 combined with the curing agent.

14. The bone cement of claim 13 which is injectable.

15. The bone cement of claim 13 which is in the form of a paste.

16. A process for making a curable bone cement comprising combining a curable polymeric binder and a filler, said binder comprising a reaction product of an aminofunctional polymer and an alkanoic acid bearing a phenolic group, said phenolic group in the binder being capable of reacting in order to cure the cement comprising phenol groups which are capable of reacting in order to cure the cement, whereby the cement is capable of curing without substantial evolution of heat on exposure to a curing agent at the body temperature of a patient in which the cement is cured.

17. The process of claim 16 wherein the curable polymeric binder is dissolved in an aqueous solvent prior to said combining.

18. The process of claim 16 wherein a second binder is combined with the binder defined in claim 16, said second binder being a reaction product of:
a carboxylic acid functional polymer, and
an alkylamine bearing a second phenolic group,
said second phenolic group in the second binder being capable of reacting in order to assist in the curing of the cement.

19. A method for curing a curable bone cement, said method comprising:
exposing the curable bone cement to a curing agent to form a catalysed bone cement; and
curing the catalysed bone cement without substantial evolution of heat;
wherein the bone cement comprises a curable polymeric binder and a filler, said binder comprising a reaction product of an aminofunctional polymer and an alkanoic acid bearing a phenolic group, said phenolic group in the binder being capable of reacting in order to cure the cement.

20. The method of claim 19 wherein the curing agent comprises a peroxidase enzyme and a peroxide.

21. The method of claim 19 additionally comprising the step of injecting the bone cement into a patient before the step of curing the catalysed bone cement.
